(19) United States
(12) Patent Application Publication
(10) Pub. No.: US 2023/0168248 A1
(43) Pub. Date: Jun. 1, 2023

(54) RAPID LATERAL FLOW ASSAY FOR VIBRIO DETECTION

(71) Applicants: CANON VIRGINIA, INC., Newport News, VA (US); UNIVERSITY OF MASSACHUSETTS, Boston, MA (US)

(72) Inventors: Michael SHIARIS, Marshfield, MA (US); Tiffany GOSINE, Newport News, VA (US); Yoichi MURAKAMI, Newport News (JP); Scott SUNDBERG, Yorktown, VA (US); Takeshi IMAMURA, Newport News, VA (US); Cristina RODRIGUEZ-QUIJADA, Framingham, MA (US); Michael TLUSTY, Melrose, MA (US); Kimberly HAMAD-SCHIFFERLI, Arlington, MA (US)

(21) Appl. No.: 17/997,635

(22) PCT Filed: Apr. 30, 2021
(86) PCT No.: PCT/US21/30286

§ 371 Date: Oct. 31, 2022

Related U.S. Application Data
(60) Provisional application No. 63/019,037, filed on May 1, 2020.

(51) Int. Cl. G01N 33/569; G01N 33/543; G01N 33/547

(52) U.S. Cl. CPC G01N 33/56911; G01N 33/54388; G01N 33/547; G01N 2333/28

(57) ABSTRACT

The present disclosure relates to methods, devices, assays and systems for rapid detection of food-borne pathogens, including Vibrios.

### PRIORITY AND INCORPORATION BY REFERENCE

The present application claims priority to U.S. Provisional Patent Application Ser. No. 63/019,037 filed May 1, 2020, the disclosure of which is incorporated herein by reference in its entirety.

### FIELD OF THE DISCLOSURE

The present disclosure relates to methods, devices, assays and systems for rapid detection of food-borne pathogens, including Vibrios in aquaculture products

### BACKGROUND

One of the greatest impediments to aquaculture production is disease. In particularly, diseases caused by Vibrio bacteria limit the potential for aquaculture production in a myriad of species including but not limited to oysters and shrimp, which are often consumed raw. Vibrios are also a major pathogenic concern beyond aquaculture as they are directly implicated in the loss of human life. The challenge of Vibrios to aquaculture is that Vibrio species are ubiquitous and at low abundance in marine environments.

Detection of Vibrios can help to ensure the safety of aquaculture products, as well as facilitating better research and management of aquaculture operations. Currently, Vibrio is detected by the culture method and/or the most-probable-number (MPN) PCR method, both of which are typically lab-based. In addition, these methods are often time-consuming due to the samples needing to be shipped to a centralized lab, as well as the time it takes to isolate, culture, detect and quantify the Vibrio.

Vibrios can limit oyster production, and rapid tests will allow for better management of hatchery production, allow farmers to manage production techniques to limit Vibrio blooms, and to also manage harvests pre- and post-bed closure. Rapid tests can also be used downstream by wholesalers and retailers for a quick, real time assessment of oyster safety, and also for researchers seeking to understand Vibrio blooms. Oysters are the most valuable marine species being farmed in the Northeastern US, with 38% of the value of the domestic marine aquaculture industry. Typical pathogens to oysters include, for example, Vibrio parahaemolyticus and Vibrio vulnificus. Detection of Vibrio in a point-of-care assay would be significant for food safety control measures, as well as researchers.

### SUMMARY OF THE DISCLOSURE

Accordingly, the present invention is based, at least in part, on the design of rapid paper- or other substrate-based immunoassays for detecting pathogens in aquaculture and other food products, such as shellfish. The immunoassays described herein can be configured for quick and easy use, and can be readable by eye and/or with assistance from a device such as a cell phone camera.

In one aspect, the specification provides a test device. The test device can include, e.g., at least one test strip, having a first end, a second end and a longitudinal axis. The test strip can include a porous carrier, e.g., nitrocellulose or filter-type paper. The first end can be configured to receive a liquid sample. In some instances, an absorbent sample pad may be disposed at and in fluid communication with the first end of the test strip, which allows sample to flow from the sample pad through the first end of the test strip. In some instances, a wick, such as a material of sufficient absorbency to draw fluid through the test strip from the first end to the second end, is disposed at the second end of the test strip. The at least one test strip can be configured as separate and individual test strips, or can be connected at the first end and separate at the second end, such that the connected test strips share a sample inlet.

The at least one test strip further includes a first detection zone disposed on the porous carrier adjacent to the first end. The first detection zone is configured to detect the presence of bacteria of the species Vibrio, and can include at least one antibody, e.g., a plurality of antibodies, that bind specifically to a specific kind of bacteria, e.g., of the Vibrio species. The Vibrio species can be any Vibrio species, such as anti-V. parahaemolyticus or anti-V. vulnificus antibodies.

The at least one test strip can further include a second detection zone disposed on the porous carrier between the first and second ends. The second detection zone is configured to determine whether any bacteria present in the sample is active, and comprises immobilized E. coli transformed with a quorum sensing plasmid specific to bacteria of the Vibrio species. In one embodiment, the second detection zone additionally comprises a chromogenic or luminescent substrate that is sensitive to the presence of AHL or alternatively, a chromogenic or luminescent substrate that is sensitive to the presence of AHL is added to the device with the sample.

The at least one test strip can further include a third detection zone disposed on the porous carrier between the first and second ends. The third detection zone is configured to determine whether any bacteria present in the sample is virulent, and can include at least one antibody, e.g., a plurality of antibodies, that bind specifically to at least one of Thermostable Direct Hemolysin (TDH) and TDH-Related Hemolysin (TRH). In one embodiment, the third detection zone can comprise two sub-detection zones, one for each of detecting TDH and TRH.

The at least one test strip optionally further includes a control detection zone disposed on the porous carrier between the first detection zone and the second end (and when present, the wick) and spatially separated from the first detection zone. The second detection zone can include at least one antibody that is sufficient for providing a positive control, for example, a plurality of antibodies that bind specifically to the Fc portion of antibodies, e.g., the same antibodies present in the first detection zone.

In some instances, the test device further includes a detection conjugate zone disposed between and in fluid communication with the sample pad and first detection zone. The detection conjugate zone can include a plurality of detection conjugates that include nanoparticles, e.g., nanospheres, such as gold nanospheres or colloids, conjugated to antibodies. It is also within the scope of this disclosure that other nanospheres could be utilized, including, for instance, latex beads, cellulose beads, and the like. The antibodies can be, e.g., antibodies that specifically bind to bacteria of the same Vibrio species as the antibodies disposed in the first detection zone. In some instances, the antibodies can be identical to those in the first detection zone. In some instances, the nanoparticles are modified at their surface with polyethylene glycol (PEG).

In other embodiments, the test device can comprise a single test strip, having a first and second end and a longitudinal axis there-between. The first end can be configured to receive a liquid sample, which sample flows from the first end to the second end of the test strip. The test strip can optionally include a sample pad at the first end and/or a wick at the second end to assist in the drawing of the sample from the first to the second end. In one embodiment, disposed between the first and second ends can be a conjugation zone or pad, a first detection zone, a second detection zone, a third detection zone (which may comprise two sub-detection zones), and optionally a control detection zone.

In another aspect, the present specification provides methods of detecting a pathogen, e.g., a bacterial pathogen such as a Vibrio, in a test sample. The method can include, e.g., providing a test device comprising:

at least one test strip comprising at least one porous carrier and having a first end comprising a sample inlet configured to receive a liquid sample, a second end, and a longitudinal axis;

a conjugation zone disposed on the porous carrier adjacent to the first end, comprising a plurality of detection conjugates comprising nanoparticles conjugated to antibodies that specifically bind to one or more targets selected from the group consisting of: (i) bacteria of the Vibrio species; (ii) Thermostable Direct Hemolysin (TDH) and (iii) TDH-Related Hemolysin (TRH);

a first detection zone disposed on the porous carrier between the conjugation zone and the second end, configured to detect the presence of bacteria of the species Vibrio, comprising a plurality of immobilized antibodies that bind specifically to bacteria of the Vibrio species;

a second detection zone disposed on the porous carrier between the conjugation zone and the second end, configured to determine whether any bacteria present is active, comprising E. coli transformed with a quorum sensing plasmid specific to bacteria of the Vibrio species; and,

a third detection zone disposed on the porous carrier between the conjugation zone and the second end, configured to determine whether any bacteria present is virulent, comprising a plurality of immobilized antibodies that bind specifically to at least one of TDH and TRH;

contacting the first end of the test device with a test sample (such as a sample comprising oyster hemolmyph) under conditions allowing the passage or wicking of the sample through the first, second and third detection zones; and

detecting the presence or absence of a positive test signal at each of the first, second and third detection zones for an indication of the presence or absence of a harmful pathogen in the test sample. An indication can be, e.g., a color change or no color change at one, two, or all of the first, second and third detection zones.

In some instances, where the device comprises a sample pad, the method can include contacting the sample pad of the test device with a test sample (such as a sample comprising oyster hemolymph) under conditions allowing wicking of the sample through the detection conjugate zone and the first, second and third detection zones.

In other embodiments, the method additional comprises determining whether the sample has tested positive for the presence of a harmful bacteria by analyzing the results detected at each of the three detection zones, wherein the sample has tested positive for the presence of harmful bacteria if any of the following results are obtained:

(1) all three tests are positive;

(2) tests for the presence of Vibrio and the activity of bacteria are positive;

(3) test for the virulence of bacteria is positive;

(4) tests for the activity of bacteria and virulence of bacteria are positive; or,

(5) tests for the presence of Vibrio and virulence of bacteria are positive.

The method can additionally comprise obtaining one or more image(s) of the at least one porous carrier, analyzing the one or more image(s) to determine whether each detection zone is positive or negative; and determining the output of the test based on the analysis of each detection zone.

In further instances, the tests at the first, second and third detection zones can be run sequentially or in parallel, and the output of each test can be monitored such that the test can be interrupted prior to completion if the test for virulence of bacteria is determined to be positive. In such instance, the test can generate an overall positive result without having to complete the remaining tests.

In still another embodiment, the present specification provides kits for detection of pathogens, e.g., in food products or environmental samples. A kit can include, e.g., a test device described herein, together with access to an application that will detect outputs from the test device and provide a determination of whether harmful bacteria are present. Such an application can be used on a smart phone, tablet, laptop, or other device.

These and other embodiments, objects, features, and advantages of the present disclosure will become apparent upon reading the following detailed description of exemplary embodiments of the present disclosure, when taken in conjunction with the appended drawings, and provided claims.

### BRIEF DESCRIPTION OF THE DRAWINGS

The accompanying drawings, which are incorporated herein and form part of the specification, illustrate various embodiments, objects, features, and advantages of the present disclosure.

FIG. 1 is a diagram depicting an exemplary test kit comprising three different types of test strips.

FIG. 2 is a diagram depicting an exemplary functionality of a test strip according to the present disclosure, having both a test line and a control line.

FIG. 3 is a diagram depicting an exemplary test kit comprising separate test strips for three assays.

FIG. 4A-B is a diagram depicting exemplary test kits comprising either a single inlet that directs sample fluid to three test strips, or a single strip test wherein the test strip comprises multiple test lines.

FIG. 5 is a flow chart depicting a Vibrio detection workflow of a device as shown in FIG. 1.

FIG. 6 is a flow chart depicting an alternate workflow based on sequential detection starting with detection of the presence of Vibrio.

FIG. 7 is a flow chart depicting an alternate workflow based on sequential detection starting with detection of TDH/TRH toxins.

FIG. 8 is a flow chart depicting a further alternate workflow that utilizes both sequential and parallel detection, starting with detection of TDH/TRH toxins.

FIG. 9 is a diagram depicting an exemplary analysis device and results process.

FIG. 10 is a diagram depicting an exemplary mechanism for measurement of test results.

FIG. 11 is a diagram and flow chart further depicting an exemplary analysis device and process.

FIG. 12 is a flow chart depicting an exemplary analysis workflow for test results.

FIG. 13 is a diagram depicting an exemplary test strip according to the present disclosure as used in a test for the presence of Vibrio.

FIG. 14A-D relates to nanoparticle (NP) synthesis and Ab conjugation. FIG. 14A is a TEM image of gold (Au) NPs. FIG. 14B is a chart showing optical absorption of bare NPs (red; bottom line) and NPs conjugated to anti-Vibrio Abs (black; top line). FIG. 14C is a picture of agarose gel electrophoresis of bare NPs (right) and NP-Ab conjugates (left). FIG. 14D is bar graphs providing DH (upper) and zeta potential (lower) of bare (right) and NP-Ab conjugates (right).

FIG. 15A is a photo of a paper sandwich immunoassay test using spiked Vibrio parahaemolyticus. FIG. 15B is a chart showing test line intensity as a function of Vibrio parahaemolyticus concentration.

FIG. 16A is a photograph showing the extraction of hemolymph from oysters. FIG. 16B is a photograph of test strips with increasing Vibrio parahaemolyticus concentration in hemolymph. FIG. 16C is a chart showing intensity of test area as a function of bacteria concentration for Vibrio parahaemolyticus (circles) and V. splendidus (squares).

FIG. 17A-C relate to negative controls for the disclosed assays. FIG. 17A is a photograph showing different negative controls compared to Vibrio parahaemolyticus: Hemolymph alone, Vibrio splendidus, E. coli and Marinomonas. FIG. 17B is a photograph showing test strips run with Vibrio splendidus in hemolymph. FIG. 17C is a chart showing line scans of test strips in 17B for E. coli (blue), Marinomonas (green), hemolymph alone (gray), Vibrio parahaemolyticus (red) and Vibrio splendidus (orange).

FIG. 18A-B are charts showing results from environmental samples from collected oysters. FIG. 18A is a bar graph showing test line intensities for different oysters that contained Vibrio parahaemolyticus in their hemolymph and an exemplary optimal test intensity cutoff value (dashed line). FIG. 18B is a graph showing the receiver operating characteristic (ROC) curve analysis for those environmental samples.

FIG. 19 is a chart of test line intensity vs. concentration, showing sensitivity of the test against a Vibrio parahaemolyticus environmental isolate.

FIG. 20 is a diagram depicting the function of a quorum sensing test.

FIG. 21A-C are photographs showing dried test strips from both with and without the use of drying protectant.

FIG. 22 is a photograph of test strips for a quorum sensing assay after different incubation times.

FIG. 23 is a chart showing intensity of the positive test spot after different incubation times for a quorum sensing assay.

FIG. 24 is a photograph of a Eppendorf tube containing an exemplary test strip for a quorum sensing assay.

FIG. 25 is a chart showing results from a quorum sensing assay using pre-prepared and stored test strips at different lengths of time in storage at 4° C.

FIG. 26 is a photograph of test strips from a quorum sensing assay that were pre-prepared and stored at different time points prior to use.

FIG. 27 is a diagram depicting the function of a TDH/TRH test.

FIG. 28 is a diagram showing the expected possible results of an assay for both TDH and TRH presence.

FIG. 29 is a depiction of expected results of a TDH/TRH assay over increasing concentrations of TDH or TRH.

FIG. 30 is a depiction of expected results of a TDH/TRH assay with respect to grayscale intensity and the resulting anticipated limit of detection.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION OF EXEMPLARY EMBODIMENT

The present disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

The present disclosure provides a simple rapid lateral flow assay test that would use the oyster hemolymph with no sample preparation. Three rapid paper-based methods are proposed to shorten this long turnaround time, which can be prepared as separate paper-based assays in a test kit, or which can be incorporated into a single paper based assay. The first method is to detect the presence of Vibrio (Liu X, et al., J Microbiol Methods. 2016 December; 131:78-84) and relies on antibody detection.

However, tests for detecting the presence of Vibrio do not distinguish between Vibrio that is active or not, which could then potentially cause a false-positive result where only inactive Vibrio is detected. Therefore, to determine how active the Vibrio is, a second method is used based on Quorum sensing (Anjali Struss, et al., Anal. Chem. 2010, 82, 4457-4463). Quorum sensing relies on detecting autoinducers secreted by bacteria during their reproductive cycles. The more active bacteria are, the greater the reproduction resulting in larger numbers of bacteria, and an increased quantity of extracellular autoinducers, the presence of which can then be detected. In gram-negative bacteria such as Vibrio, autoinducers can include acyl-homoserine lactones (AHL). Secretion of AHL starts a reaction process that leads to the downstream transcription of particular genes, including those that result in the production of biofilm (Mizan et al. Biofouling 2016; 32(4):497-509). In the presence of x-gal or other chromogenic/luminescent substrates that are sensitive to AHLs, such as Beta-Glo, a visible signal such as a blue coloration will confirm the presence of AHL, and confirm that the bacteria are active.

Further, even if the Vibrio is active as shown, for instance, by the presence of AHL, the Vibrio may not be virulent. Therefore, a third method is utilized to detect if the Vibrio is pathogenic or not by measuring the hemolysin by means such as TDH (Thermostable Direct Hemolysin) and TRH (TDH-Related Hemolysin) released from Vibrio (Junko Sakata, et al., International Journal of Food Microbiology 264(2018) 16-24), through the use of antibodies to the TDH and TRH proteins.

The three test assay of the present disclosure provides a reduction in time to result as the tests can be performed onsite or in the field, rather than having to be sent to a lab. Early detection of Vibrio before it becomes pathogenic can help farmers make better decisions on how to protect their aquaculture crop (e.g., oysters) and potentially stop foodborne outbreaks before they occur. Being able to detect Vibrio as it becomes pathogenic will help ensure the safety of aquaculture products, as well as allowing better research and management of aquaculture operations.

Test Strips

FIG. 1 shows a non-limiting, exemplary structure of a test kit according to the present disclosure. In this exemplary configuration, the test kit has three test strips, each of which contains one type of test: (1) Lateral flow test strip to detect Vibrio, (2) Quorum sensing test strip to detect activeness of Vibrio, and, (3) Lateral flow test strip to detect pathogenicity (TDH (Thermostable direct hemolysin)/TRH (TDH-related hemolysin)). In an alternative embodiment, a single lateral flow test strip could have at least three separate reaction areas, each of which contains a single test, for instance, an immunoassay to detect the presence of Vibrio, a quorum sensing test to determine the activeness of Vibrio, and an immunoassay to detect pathogenicity. In one embodiment as shown in FIG. 1, each test strip has three areas. The first area is an inlet area or sample pad to receive a sample fluid. In one embodiment, the sample fluid can comprise extracted oyster hemolymph. In a further embodiment, the sample fluid can be pooled from multiple specimens to be tested, or the sample fluid can be obtained from the environment in which the seafood has been harvested, such as sea water.

The second area of each test strip is a conjugation pad. The conjugation pad will only be present on the test strip for immunoassays. The conjugation pad serves as a storage for the antibody-labelled nanoparticles. As the sample fluid travels through the test strip, the antibodies will bind to the target analyte if present in the sample fluid.

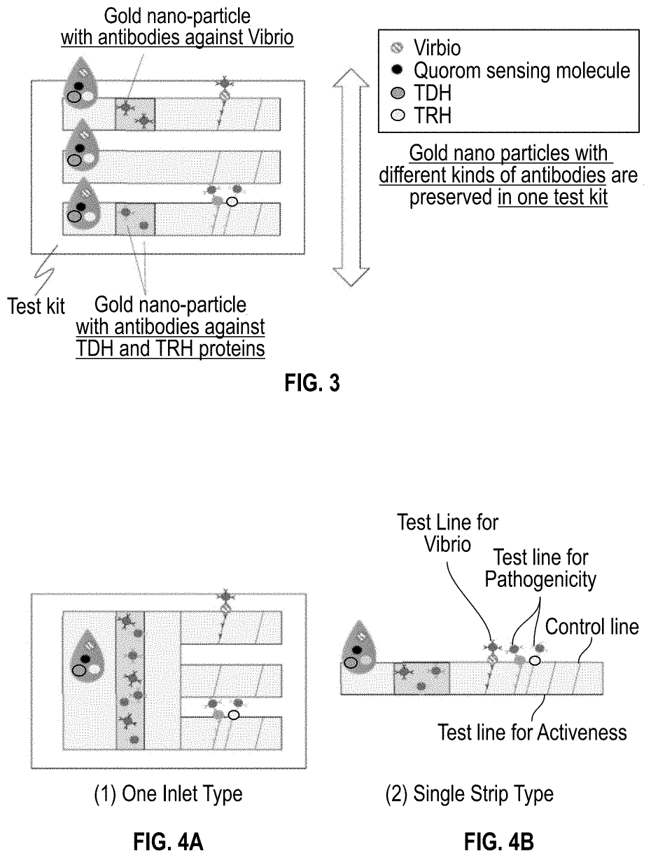

The third area of each test strip is the particular test or reaction area. For instance, the test areas can be configured to provide a test of the presence of Vibrio, the activeness of a Vibrio, and the pathogenicity of a present Vibrio. In the instance of a test strip for pathogenicity, a test area can be present for one or both of TDH and TRH. For those tests that are immunoassays, the test area will contain antibodies to the test target analyte that are immobilized on the test strip, for instance, in a line or other pattern on the test strip. Accordingly, if the target analyte is present, it will be bound via the immobilized antibody to the test strip at the test or reaction area. As the target analyte may have been bound to an antibody-labelled nanoparticle as it passed through the conjugation pad, the target analyte can be both immobilized at the test or reaction area, and be further conjugated to an antibody-labelled nanoparticle. Further details of an additional exemplary configuration of one of the test strips can be found in FIGS. 3 and 4B.

A control area may or may not be present. If present, the control area will provide a positive indication that the fluid sample has travelled successfully through the test strip. Consequently, the presence of the control area is optional, as visible wetting of the strip may serve as a passive control. If a specific control area is present, once the sample fluid passes through the control area, a visible signal will be provided. For instance, an indicated color or pattern (such as a dot or line) will be visible. As a control area, the visible signal must occur in when used in samples that are both positive and negative for the bacterial tests. The control area can contain a substance that will trigger a visible signal once the sample fluid passes through the control area. In one exemplary embodiment, the control substance can be an antibody, such as an anti-rabbit IgG (Fc) antibody. Those of skill in the art will readily recognize other control substances that can be used.

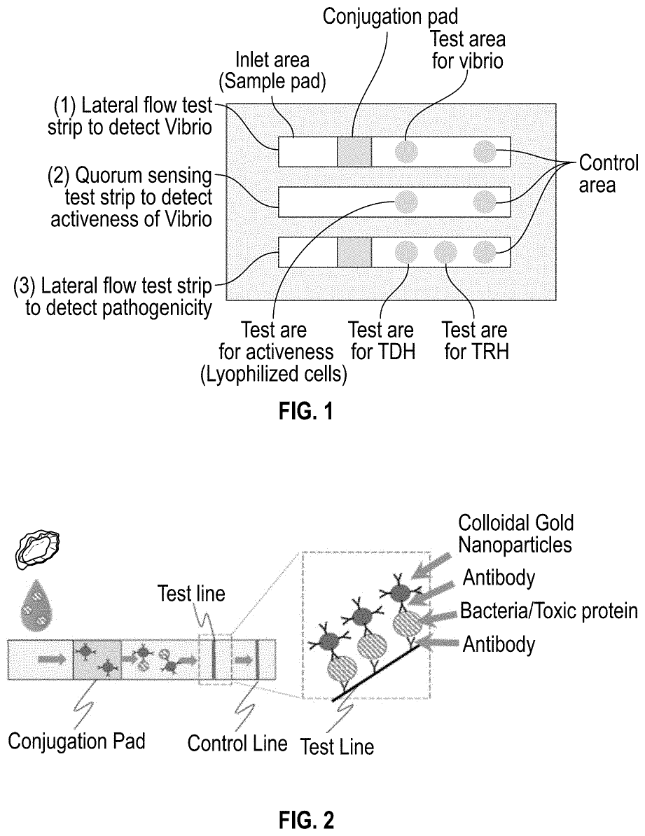

FIG. 2 provides a further exemplary configuration of a test strip in which the detection areas comprise a line perpendicular to the longitudinal axis of the test strip. In the instance of FIG. 2, a sample area to the left of the test strip is followed by a conjugation pad containing colloidal gold nanoparticles conjugated to antibodies. As the liquid sample moves through the test strip, bacteria and/or toxic proteins in the sample are bound to the antibody such that the bacteria and/or toxic proteins are bound to the gold nanoparticles. The bound bacteria and/or toxic proteins continue travelling through the test strip to the test line, which comprises bound antibodies of the same type as on the gold nanoparticles. The bound bacteria and/or toxic proteins also bind to the immobilized antibodies, causing an accumulation of gold nanoparticles to form at the test line, thereby providing a visible indicator that the test is positive. Beyond the test line can be an optional control line which also comprises bound antibodies of a type that will provide a positive result regardless of the presence of bacteria in the sample, for instance, an anti-rabbit IgG (FC) antibody.

FIG. 2. additionally provides an enlarged detail of a positive test line, depicting the test line with immobilized antibodies, which have attached to the bacteria and/or toxic protein that were previously conjugated to the gold nanoparticle-antibody conjugates.

FIG. 3 provides test kit which includes three separate test strips, each of which is prepared for a different assay or test. In the top test strip, the conjugation pad includes gold nano-particles with antibodies against Vibrio. The middle test strip is configured for a quorum sensing test to determine activity. In the bottom test strip, the conjugation pad includes gold nano-particles with antibodies against TDH and/or TRH. As shown in this FIG. 3, the bottom test strip is depicted as having two sub-detection zones, each of which is a test line for either TDH or TRH. In this instance, by separating the detection zones for the two toxic proteins, it is possible to determine which one is present, whereas in a configuration where the detection zone contains antibodies for both TDH and TRH, if a positive result is obtained, it cannot be immediately determined which of the toxic proteins (or both) is present.

FIGS. 4A and 4B provide additional alternate configurations of the present disclosure. FIG. 4A provides a test device wherein the test device has been cut such that the first end of the porous carrier has a width that is greater than that of three individual test strips, such that a single sample inlet can be shared. The second end of the porous carrier has been cut such that three separate second ends are present. That is, if starting from a rectangular shaped porous carrier, the first half of the porous carrier remains intact, while the second half is cut to separate the second half into three separate and distinct strips, which are attached to each other via the first half of the porous carrier. As shown, the sample inlet can then be shared by all three second ends of the porous carrier. In addition, the conjugate pad can optionally be positioned such that it stretches perpendicular to the longitudinal direction across the first half of the porous carrier, such that the conjugate pad is also shared by all of the three second ends of the porous carrier. Each second end of the porous carrier can then be configured as if it was an individual test strip with respect to the placement of the detection zone(s) between the conjugate pad and the second end. Optionally, a control line can be included in the control detection zone.

FIG. 4B provides an additional alternate configurations of the present disclosure wherein a single test strip contains the sample inlet, conjugation pad, test lines for the presence of Vibrio, pathogenicity/virulence of the bacteria (including sub-detection zones, if desired), a test line for activeness of the bacteria, and an optional control line.

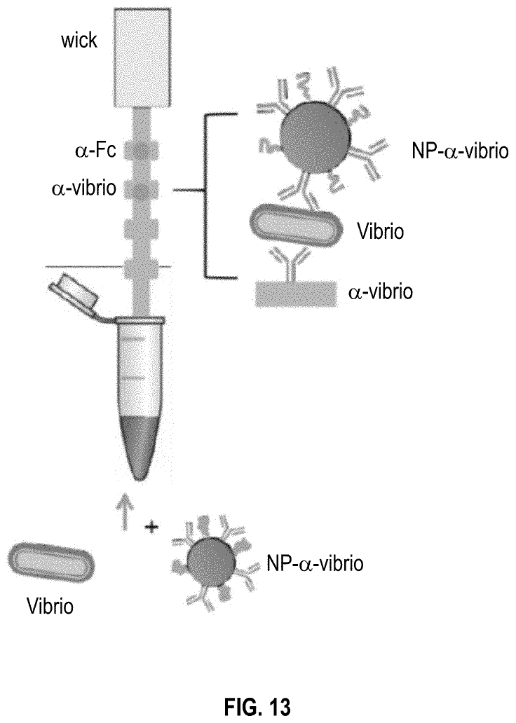

Additional embodiments of the present disclosure include the optional placement of a sample pad at the first end of the porous carrier. The sample pad may be an absorbent material which may be disposed at and in fluid communication with the first end of the test strip, to receive the liquid sample and which allows sample to flow from the sample pad through the first end of the test strip. In some instances, a wick, such as a material of sufficient absorbency to draw fluid through the test strip from the first to the second end, is disposed at the second end of the test strip. Such a wick can be as depicted in FIG. 13.

It is noted that although “test line” is used herein, the placement of the detection molecules (antibodies and/or transformed E. coli) in the detection zones can be done in any desired configuration. These can take the form of lines, spots, or other patterns.

Lateral Flow Immunoassay Line to Detect Vibrio Itself

In one exemplary embodiment, the test for the lateral flow immunoassay comprises three areas as discussed above. The first area is the sample pad to receive the sample fluid. The second area is the conjugation pad where conjugations of gold nano-particle and polyclonal anti-Vibrio antibody are placed. The same antibody is also immobilized in the test area to capture any Vibrio present in the sample. For the control area, an antibody such as an anti-rabbit IgG (Fc) antibody is placed to capture the conjugates. When the sample fluid is dispensed to the sample pad, the pad absorbs fluid and fluids travel through the test strip to the downstream areas. The fluid reaches the conjugation pad, and carries these conjugated nano-particles placed in the conjugation pad further downstream in the test strip. If the fluid flows successfully downstream through the test strip, the control conjugations are captured by the antibodies at the control area and shows a color due to the aggregated gold nano-particle. If Vibrio is present in the sample fluid, the nano-particle conjugations having antibodies specific to Vibrio attach to the Vibrio and once the Vibrio has traveled through the test strip to the test area, the Vibrio is captured by the antibodies at the test area. The accumulation of the gold nano-particles will then cause a color to become visible, indicating the positive result.

Quorum Sensing Line to Detect Activeness of Vibrio

The quorum sensing test detects bacterial quorum sensing signaling molecules, such as N-acylhomoserine lactones (AHLs). AHLs are produced by a luxl enzyme and are generally employed by Gram-negative bacteria for their cell-cell communication. This allows the controlled expression of specialized genes, such as those involved in biofilm formation and production of virulence factors, in a population-density-dependent manner. Escherichia coli cells are transformed with a quorum sensing plasmid which has the necessary gene sequences to enable sensing the presence of AHL. In the case of Vibrio parahaemolyticus, this includes luxbox and luxR. When the AHL is present, it results in the expression of the reporter protein, β-galactosidase. Since the β-galactosidase cleaves X-gal to form an insoluble blue product, such that the amount of the reporter protein produced can be measured using a colorimetric-based detection. In this test strip, lyophilized E. coli cells transformed with a quorum sensing plasmid as described above are placed on the test area. When the sample fluid is introduced to the inlet area and passed through the test area, the blue color gradually develops. The intensity of the color increases as the concentration of the quorum sensing molecules increases, thereby providing an indication of the activity of the Vibrio. A test that is positive for Vibrio will show a visible color at the site of the quorum sensing test area.

Lateral Flow Immunoassay Line to Detect Pathogenicity of Vibrio (TDH/TRH)

Vibrio parahaemolyticus produces multiple toxins, including thermostable direct hemolysin (TDH) and TDH-related hemolysin (TRH). Thus, an indication of the pathogenicity of Vibrio is the presence of TDH or TRH. An immunoassay to one or both of TDH and TRH can be prepared using the basic structure as the previously described immunoassay to detect Vibrio.

Using an exemplary test strip as provided in FIGS. 1 and 2, the TDH/TRH immunoassay has three areas: a first area of a sample pad to receive the sample fluid, a second area of a conjugation pad where conjugations of gold nano-particle and anti-TDH and/or anti-TRH antibodies are placed. The same antibody is also immobilized in the test area to capture any TDH and/or TRH proteins present in the sample. For the control area, an antibody such as an anti-rabbit IgG (Fc) antibody is placed to capture the conjugations, with conjugations of matching gold nano-particles and anti-rabbit antibody also included in the conjugation pad. When the sample fluid is dispensed to the sample pad, the pad absorbs fluid and fluids travel through the test strip to the downstream areas. The fluid reaches the conjugation pad, and carries these conjugated nano-particles placed in the conjugation pad further downstream in the test strip. If the fluid flows successfully downstream through the test strip, the control conjugations are captured by the antibodies at the control area and shows a color due to the aggregated gold nano-particle. If TDH and/or TRH is present in the sample fluid, the nano-particle conjugations having antibodies specific to those protein(s) will attach to the proteins and once the protein(s) have traveled through the test strip to the test area, the TDH and/or TRH is captured by the immobilized antibodies at the test area. The accumulation of the gold nano-particles will then cause a color to become visible, indicating the positive result. In those instances where tests for both TDH and TRH are desired, two separate test areas can be provided in the test strip, each one having immobilized antibodies to one of the toxins of interest.

Test Work Flow

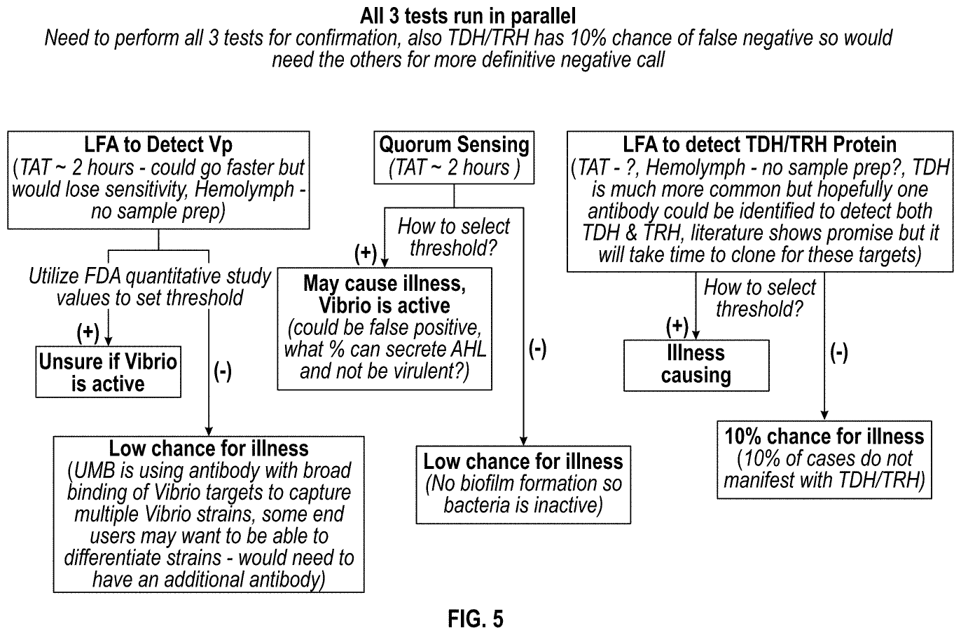

As described previously, one exemplary embodiment of the present disclosure is to combine these three test methods in one device as shown in FIG. 1, 3 or 4A. FIG. 5 provides a parallel workflow that would be suitable for use with any of these configurations, in which the three assays (immunoassay to detect Vibrio, quorum sensing to determine activity, and immunoassay to detect toxic proteins) are run at the same time, which each providing a positive or negative result. When following the parallel workflow of FIG. 5, the overall output of the test can be determined pursuant to the possible outcomes shown in Table 1.

TABLE 1

| | Result | | | | | | | |
|---|---|---|---|---|---|---|---|---|
| Detection of Vibrio | + | + | + | − | − | − | − | + |
| parahaemolyticus. | | | | | | | | |
| Quorum Sensing of Vibrio | + | + | − | − | − | + | + | − |
| parahaemolyticus. | | | | | | | | |
| Virulence of Vibrio | + | − | − | − | + | + | − | + |
| parahaemolyticus. | | | | | | | | |
| Judgement | + | +* | − | − | +** | +*** | − | + |

*May be 10% of positive by TDH/TRH negative
**Low probability of being caused by Vibrio parahaemolyticus
***Positive from another species

Based on the judgement table above, any time the virulence test is positive, the overall test result is also positive. Although, for those instances in which the detection of Vibrio is negative and the virulence is positive, it can be inferred that the virulence test has shown a positive result based on the presence of a bacteria other than a Vibrio.

In the workflows of the present invention, a decision or judgement of harmful is equated with a risk of illness caused by the sample, and a decision or judgement of not harmful is equated with no concerns for illness caused by the sample.

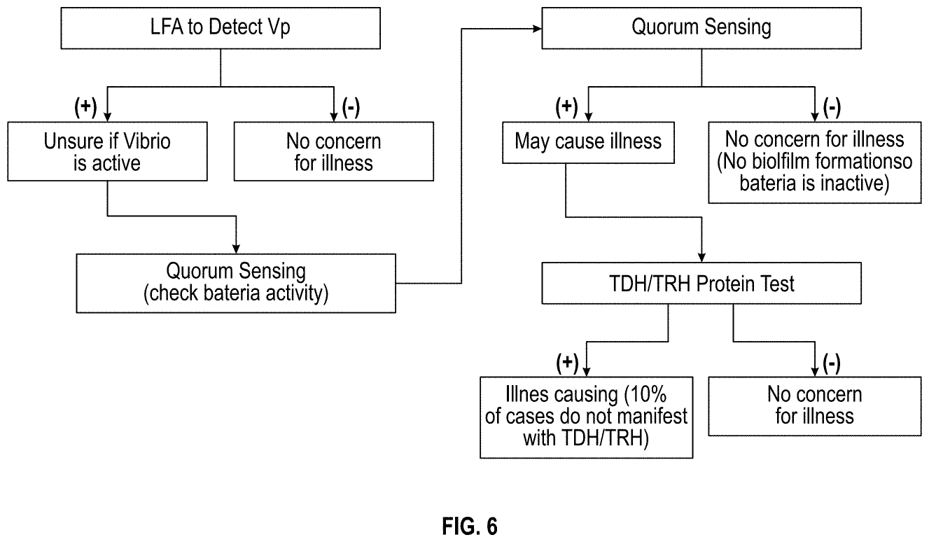

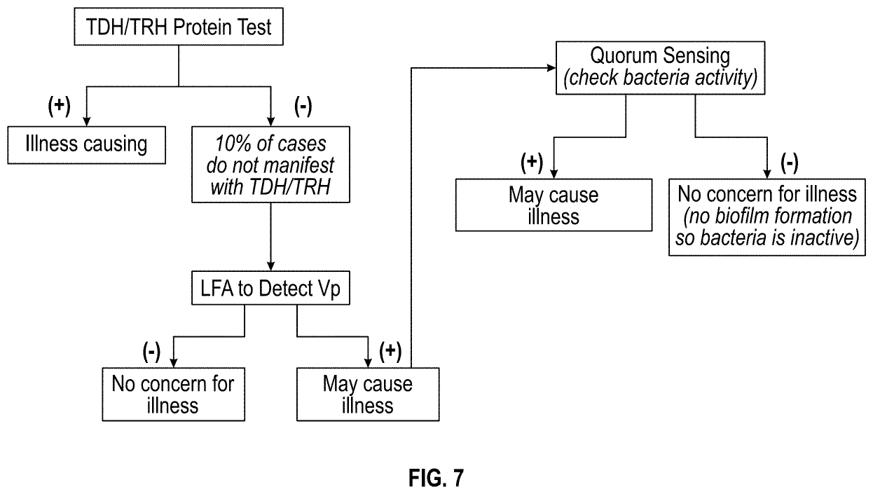

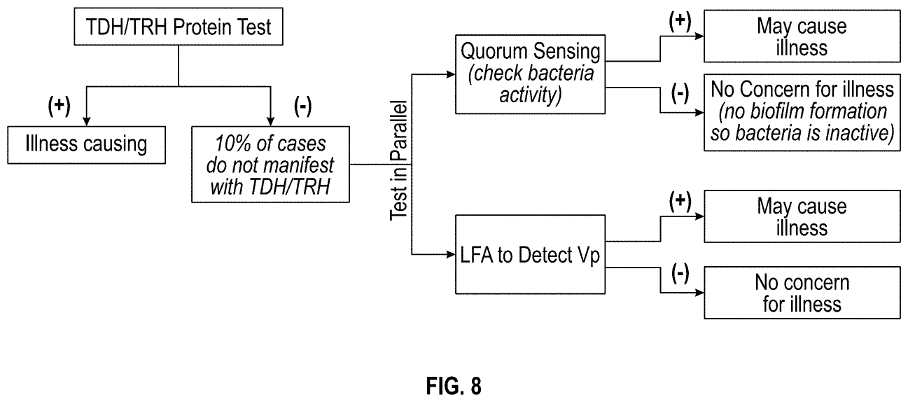

Another method for testing would be to use a sequential workflow shown in FIGS. 6 and 7 or a TDH/TRH driven workflow shown in FIG. 8. These alternate workflows could improve the test reliability by lowering the possibility of false-negatives just by detecting TDH/TRH and lowering the possibility of false-positives by detecting the presence of Vibrio and activeness of Vibrio for those 10% of cases in which the Vibrio may not be producing TDH/TRH.

In FIG. 6, a workflow is provided whereby the initial test performed is the immunoassay to detect Vibrio. In this instance, the testing would only continue if the initial Vibrio test rendered a positive result that Vibrio was present in the sample. A negative result would stop all further testing in the workflow and the sample would be considered not harmful. In the event Vibrio was found to be present, the testing workflow would continue to the quorum sensing test, in order to determine whether the Vibrio is active. A negative quorum sensing test, which typically correlates with no presence of a biofilm, would stop the testing workflow and the sample would be considered not harmful. In the event of a positive quorum sensing test, the workflow would continue to the last test, where the sample would be tested for the presence of TDH and/or TRH. In this instance, a negative result for presence of TDH and/or TRH would provide a not harmful determination for the sample.

In order to perform any of the workflows provided in FIGS. 5-8, it is understood that the test device can be configured to match the workflow. For instance, the workflow of Example 5, where all three tests are run in parallel, would match with the test device configuration of FIG. 1, 3 or 4A. The configuration of the test devices shown in FIGS. 1 and 3 include test strips that may be separate such that the tests can be manually initiated in any desired order. This means that the configurations of FIGS. 1 and 3 could be use with any of the workflows provided herein as the user would simply follow the workflow in initiating the tests.

Similarly, those test device configurations provided herein such as in FIG. 4B are ideally suited for running the various tests in sequence. The order of the detection zones can be configured to match the desired workflow, as seen for instance, in FIGS. 6 and 7, both of which are run fully in series, but in which the tests are performed in different orders.

FIG. 7 provides an alternate workflow wherein the sample would first be tested for the presence of TDH and/or TRH. A positive result would stop the workflow and the sample would be deemed harmful. A negative result for the presence of TDH and/or TRH would be followed by the immunoassay to detect the presence of Vibrio. A negative result for the presence of Vibrio would cause the workflow to cease, and the sample would be considered not harmful. A positive indication of the presence of Vibrio would be followed by the quorum sensing test to determine if the Vibrio was active. A positive test for activity leads to the sample being deemed harmful, while a negative test will indicate the sample is not harmful.

FIG. 8 provides a further alternate workflow that combines both sequential and parallel testing. This workflow begins with the testing the sample for virulence, wherein a positive result stops the workflow and the sample is deemed harmful. If the virulence test is negative, the test for the detection of Vibrio and the quorum sensing tests are run in parallel. The results of the Vibrio detection and quorum sensing tests can then be confirmed with the decision matrix in Table 1 to determine the final result.

Detection and Analysis System

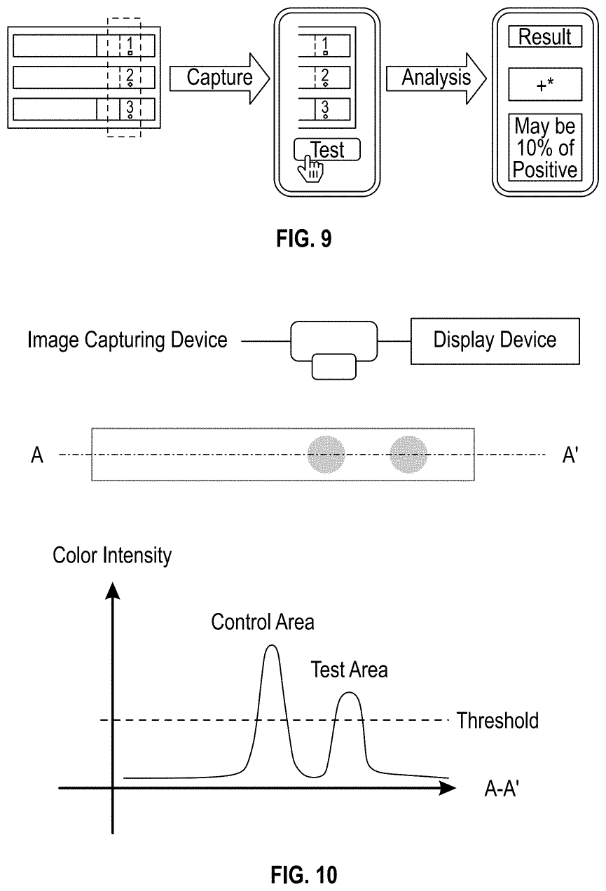

FIG. 9 provides an exemplary method and system for analyzing the results of the three tests (Vibrio detection, quorum sensing and virulence) and for determining a final judgement or decision for the sample. An image of the test device's detection areas (shown as a dotted line) can be captured by a device. The device can be a cell phone, tablet, camera, or other imaging device. An analysis application can be run by the same device or by a control system that is connected to the image capturing device, in which each detection zone is determined to be positive or negative, and a decision table such as Table 1 can be utilized to provide a final determination. As certain determinations may have additional information that is useful to the user (for instance, that in 10% of illness causing instances, the TDH/TRH is negative, that there is low probability of the results being caused by Vibrio parahaemolyticus, or that the test is likely positive from another species), the resulting visual display of the result can also include this information.

FIG. 10 shows an exemplary mechanism by which detection and measurement can occur. As previously provided, an image capturing device can be used. In addition, use of a measurement system is provided in order to reduce error, including human error when determining the results of the Vibrio test(s). There are several methods to judge a positive/negative result for each test strip or for each detection zone. One method is to obtain an image of the detection zone and then measure the color intensity of each test strip compared with the threshold intensity for each. Threshold intensities can be separately determined for each test, or can the same for each one. In one example, once each reaction is completed, digital images for each test strip are acquired and converted to gray scale images. Then the average gray values of each test area are calculated and compared with each threshold value. If the measurement value is above the threshold, the system judges the test result as positive. If not, the system judges the test result as negative.

Result Analysis Workflow

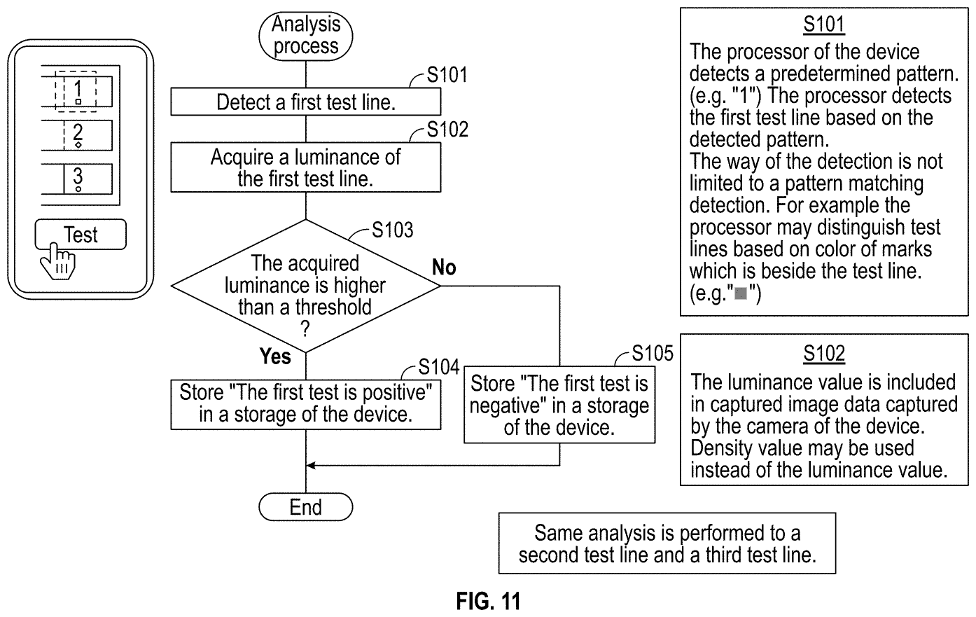

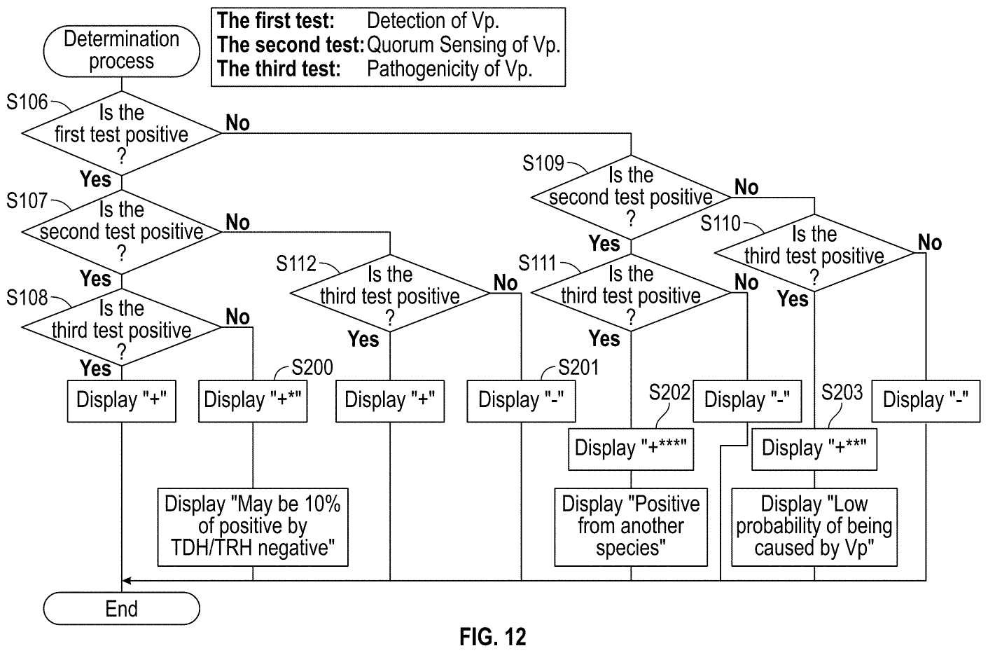

FIG. 11 provides an exemplary analysis process, and FIG. 12 shows an exemplary workflow specific to Vibrio analysis. In FIG. 11, following completion of the tests, an image of the detection zones is obtained, and the analysis is started. In Step S101, a first test line is detected. S102 acquires a luminance of the first test line. Step S103 asks whether the acquired luminance is higher than a set threshold. If yes, S104 stores “the first test is positive” in a storage media of the device, and the analysis process for this first test ends. If S103 is no, S105 stores “the first test is negative” in a storage media of the device, and the analysis process for this first test ends. The analysis process is then repeated for each remaining test. It is noted that in S101, the processor of the device detects a predetermined pattern in order to determine which detection zone to analyze. This could use pattern matching with an alpha/numeric value, or it could be configured such that the processor can distinguish between detection zones based on a colored mark that is in the vicinity of the detection zone.

Once the system judges each test strip, overall judgement will be shown in the display according to the judgement table (Table 1). Though the judgement can be done after all test strips shows results as shown in FIG. 5, the system can show the judgement along the sequential workflow (FIGS. 6-8). This sequential workflow can reduce the turn-around time from inputting sample fluids to showing results.

FIG. 12 provides an analysis workflow for a Vibrio test wherein the first test is the detection of Vibrio, the second test is the quorum sensing for activity, and the third test is the pathogenicity or virulence of the Vibrio. In S106 asks whether the first test is positive. If yes, S107 asks whether the second test is positive. If Yes, the test system displays a positive, and the analysis ends. If instead the answer to S106 is no, S109 asks whether the second test is positive. If no, S110 asks whether the third test is positive; if yes, S111 asks if the third test is positive. Similarly, if the answer to S107 is negative, S112 then asks if the third test is positive. Based on the answers to the results of each test, the system returns a displayed judgement, and in the instances of S200, S202 and S203, can additionally provide information beyond the positive or negative judgement.

The analysis workflow of FIG. 12 can provide numerous benefits in comparison to the current state of the art, which includes only a test for the presence of Vibrio. The benefits are summarized in Table 2, which references the steps depicted in FIG. 12.

TABLE 2

| | Test: Presence | 3 Test Analysis of | |
|---|---|---|---|
| Step | of Vibrio only | present disclosure | Benefit |
| S200 | Positive | Positive (but | The possibility of |
| | | possibility is low) | being positive is low. |
| S201 | Positive | Negative | The oyster is safe. |
| S202 | Negative | Positive | The oyster is not safe |
| | | | because of a bacteria |
| | | | other than Vibrio. |
| S203 | Negative | Positive | There is a possibility |
| | | | of being positive, but |
| | | | the possibility is low. |

The three-test analysis provided herein related workflows as seen in FIG. 12 therefore provide additional information to the user that is unavailable when only the presence of Vibrio is assayed, resulting in additional opportunities to find illness causing samples, and to avoid the unnecessary waste of food and expense that may result from samples that test positive for Vibrio, but which are not likely to cause illness.

### EXAMPLES

### Example 1: Lateral Flow Immunoassay to Detect Vibrio

Gold Nanoparticle Synthesis and Characterization

Gold nanoparticles (AuNPs) were synthesized using citrate reduction of gold (III) chloride using the technique described in Turkevich et al. (Discussions of the Faraday Society 1951, 11, 5-75). In short, 1 mL of a 6.8 mM sodium citrate solution was added to a 50-mL solution of HAuCl4 at 0.25 mM while boiling. The solution was left to boil for 15 min while stirring to allow the AuNPs to form, and then cooled down to room temperature with continued agitation.

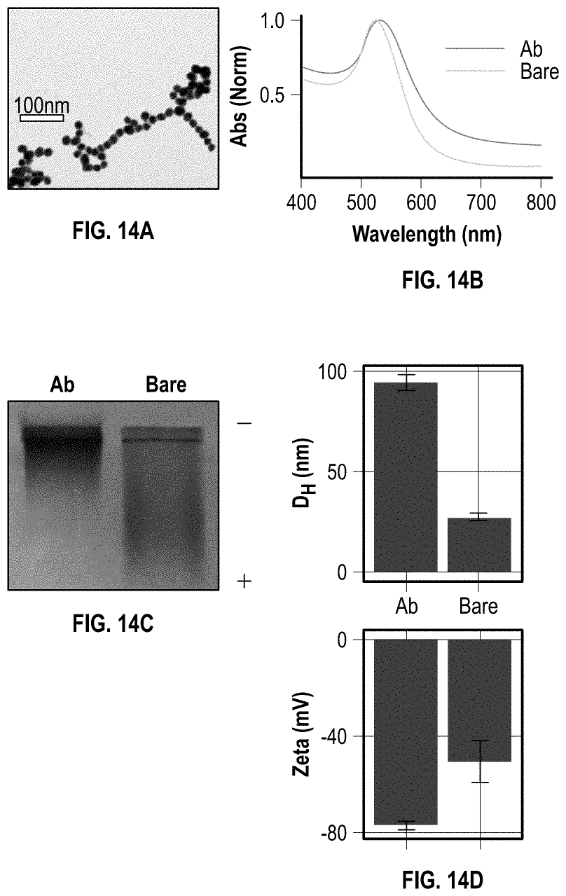

The optical absorption properties of the AuNPs were characterized by UV-vis spectroscopy (Agilent Cary 5000 UV-Vis NIR). The morphology of the AuNPs was verified by TEM (FEI Tecnai G2 at 120 kV) and ImageJ was used to determine their size. NPs had a mean diameter of 18.8±2.5 nm (FIG. 14A). The Nanoparticle Analyzer SZ-100 from Horiba was used to measure the hydrodynamic diameter (DH) (27.5±1.8 nm (FIG. 14D)) and the zeta potential (ζ) of the bare and Ab-conjugated AuNPs, which showed that the NPs had a negative charge of −50.7±8.7 mV (FIG. 14D). Optical absorption spectroscopy determined that the nanoparticles had a surface plasmon resonance (SPR) peak wavelength of 524 nm (FIG. 14B).

Antibody Conjugation

Bioconjugation of the antibody to AuNPs was performed by electrostatic binding. Aliquots of 1 mL of the AuNPs synthesis solution were first centrifuged at 12000 rcf for 12 min and the pellet was resuspended in a mixture of 100 μL of 40 mM HEPES at pH 7.7 and 300 μL of MilliQ water. Then, 5 μL of a 1 mg/mL anti-Vibrio antibody solution reconstituted in phosphate buffer 10 mM at pH 7.4 was added to the solution and agitated overnight at room temperature to avoid the binding of the antibody to the AuNP surface. 10 μL of 0.1 mM mPEG was added to avoid nonspecific binding on the AuNP (Hristov et al. ACS Applied Materials & Interfaces 2020, 12(31):34620-34629; Rodriguez-Quijada et al. ACS Nano 2020, 14, 6626-6635). The solution was left under agitation at room temperature for 15 min. Excess reagents were separated by centrifuging the pellets at 8000 rcf and the resulting ˜25 μL pellet was used in the dipsticks. Agarose gel electrophoresis was also used to confirm the antibody conjugation by running a mixture of 8 μL of concentrated AuNPs with 4 μL of 50% glycerol in a 1% agarose gel. The conjugation was confirmed with a higher retention of the conjugates when run in the gel as a result of their increased size (FIG. 14C). This size increase was also confirmed by DLS, leading to bioconjugate D_{H} of 94.5±3.4 nm (FIG. 14D). Also, a decrease of the bioconjugate charge was observed compared to the bare nanoparticles, with a zeta potential of −77.1±1.8 mV (FIG. 14D). The bioconjugate SPR peak resulted in a red shift and a slight broadening of the peak, also suggesting a successful NP conjugation (FIG. 14B).

Vibrio Culture

V. parahaemolyticus ATCC® 17802™ was grown on thiosulfate citrate bile salts sucrose agar (TCBS) plates. A single colony was inoculated in 5 mL of saline lactose broth (SLB) and grown at 30° C. overnight. To better control bacterial growth, an inoculum from this solution was taken with an inoculation loop and added to 5 mL of fresh sterile SLB broth and left for 5 h at 30° C. to reach desired concentration. To calculate the concentration of the sample, a 1:100.000 dilution was made in sterile 1×PBS and 50 μL of the diluted sample was spread in SLB plates left to grow overnight at 30° C. Counting was done in triplicate. Samples were kept at 4° C. to prevent further growth until they are run in the rapid tests.

Hemolymph Extraction

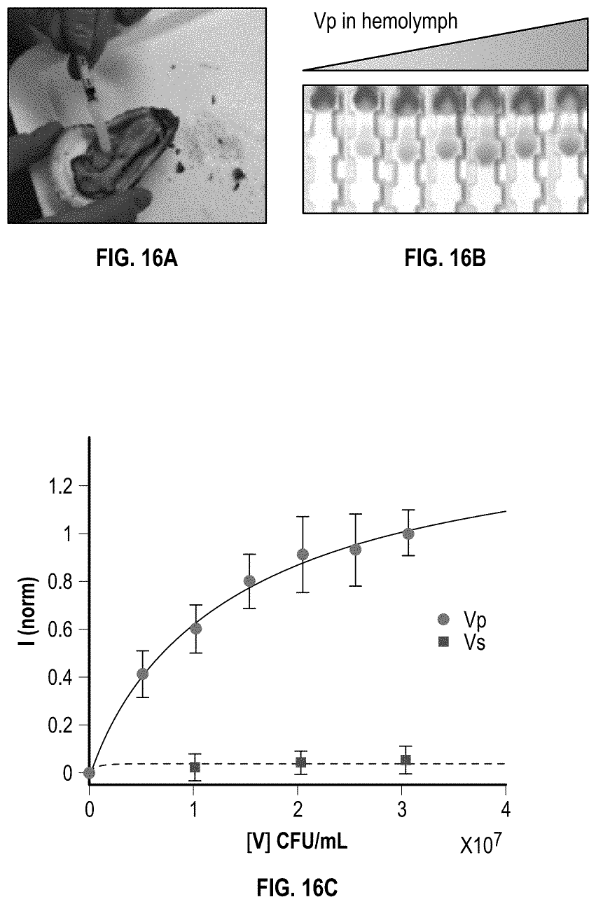

Hemolymph was obtained from oysters, which were either purchased locally at a market or obtained from local coastal areas. Oysters were shucked and hemolymph was extracted with a syringe (FIG. 16A). For spiked samples, the hemolymph was sterilized by filtering it through a sterile 0.22-μm pore-size filter and kept at −20° C. until use.

Negative Control Isolation from Environmental Samples (V. splendidus and Marinomonas)

Hemolymph extracted from market oysters was directly plated on TCBS and SLB agar and incubated for 24 hours at 37° C. Isolated colonies were streaked for isolation of pure culture on TCBS and SLB agar. Pure colonies were inoculated into 5 mL SLB broth and incubated overnight at 37° C. Nucleic acid extractions were performed on SLB cultures using Qiagen DNeasy UltraClean® Microbial Kit.

Vibrio parahaemolyticus Isolation from Environmental Samples

Hemolymph extracted from local coastal oysters was enriched for Vibrio isolation following a procedure modified from Hartnell et al. (International Journal of Food Microbiology 2019, 288, 58-65). Briefly, 300 μL hemolymph was added to 2.7 mL alkaline peptone water (APW) and incubated for 6 hours at 37° C. Following incubation, 300 μL of the initial culture was inoculated into 2.7 mL APW and incubated for 18 hours at 37° C. Enrichments were streaked for isolation of pure culture on TCBS agar. Individual colonies characteristic of Vibrio parahaemolyticus growth (dark blue-green) were picked into SLB broth and incubated at 37° C. overnight. Nucleic acid extractions of SLB cultures were performed using PrepMan™ Ultra Sample Preparation Reagent (ThermoFisher).

Isolates were archived in 75% SLB broth/25% glycerol and stored at the −80° C. Archives were regrown by streaking on TCBS plates and followed the same protocol with the ATCC Vibrio parahaemolyticus to let them grow to the concentrations used to run them in the immunochromatography tests.

PCR and Sequencing

Bacterial isolates were identified using PCR amplification and sequencing of the 16S rRNA gene and the gyrase B subunit gene (gyrB), using primers 8F (5′-CCTACGGGAGGCAGCAG), 534R (5′-ATTACCGCGGCTGCTGG) and Up1E (5′-GAAGT CATCA TGACC GTTCT GCAYG CNGGN GGNAA RTTYR A), UP2AR (5′-AGCA G GGTAC GGATG TGCGA GCCRT CNACR TCNGC RTCNG YCAT) respectively (Muyzer et al. 28; Green et al. 29). Sequencing was performed by Eton Biosciences, Boston, Mass. Identification of all isolates was determined by 16S rRNA and gyrB sequence matches using BLASTn (NCBI) against the nucleotide collection (nr/nt) database.

Paper Immunoassays

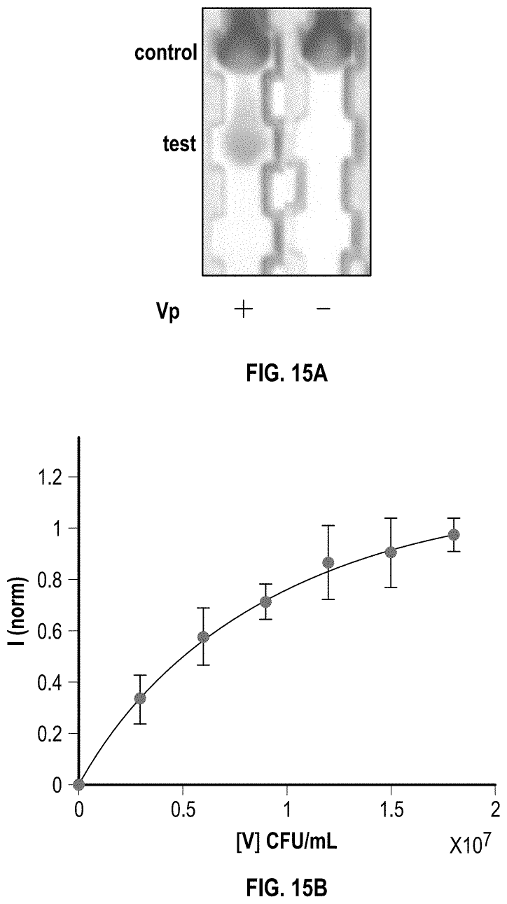

CN140 nitrocellulose strips were laser cut (LaserPro Spirit LS) at power 85% and a speed of 100%. Strips were attached to the absorbent pad with the help of a backing paper. Antibodies were spotted manually in 0.3 μL aliquots to obtain 1.2 μg of Ab spotted on the test line. For the positive control, an anti-rabbit Fc specific antibody was immobilized on the control line. A blocking step was added to prevent false positives. Strips with the spotted antibody were left to dry at least 30 minutes before adding them to a 1.5 mL microcentrifuge tube with 50 μL BSA solution at 1 mg/mL and left to dry at room temperature overnight. Tests were run in sequential steps to get the best results by immersing the strip in different solutions and letting the solution migrate through the strip by capillary action. First, a solution containing 12 μL of a sucrose/tween mixture (4 μL 50% w/v sucrose in water and 8 μL of 1% v/v Tween 20 in 1×PBS) and 30 μL of bacteria spiked in sterile hemolymph or PBS 1×was run. Then, the strips were washed with 50 μL PBS-Tween 20 0.1% (PBST) to avoid non-specific binding of the bacteria with the nitrocellulose. Once the strip was washed, a solution containing 12 μL of sucrose/tween, 3 μL of conjugated NPs and 30 μL of Human Serum was run to prevent false positives (de Puig et al. Bioconjugate Chemistry 2017, 28, 230-238). A final step with 50 μL of PBST was run to wash the nitrocellulose from nonspecific binding. The strips were left to dry at room temperature and scanned for quantitative analysis. ImageJ was used to measure the gray intensity of the test area. Results are shown in FIG. 15A-B. An intense spot at the test area was observed when Vibrio parahaemolyticus was present in the sample (FIG. 15A). This indicated that the NP-Ab conjugates were retained at the test line due to the specific interaction of both the capture and detection antibodies with the bacteria. The successful generation of this sandwich immunoassay showed that the adsorbed antibody was not displaced from the NP surface by the PEG backfill or the PC formation. When PBS without Vibrio parahaemolyticus present was run, no spot appeared at the test line, suggesting that the Vibrio parahaemolyticus binding was not due to specific interactions of the probes with the immobilized antibodies (FIG. 15A). A second colored spot appeared at the control line due to the binding of the control antibody to the anti-Vibrio Ab conjugated to the NPs and therefore validating the test. This was indicative of a proper flow of the NPs as well as the correct Ab conjugation of the Au NPs while migrating through the test.

To determine the LOD of the test in 1×PBS, the strips were run at different Vibrio parahaemolyticus concentrations ranging from 0 to 1.8×10⁷ CFU/mL. Using image analysis, test line signal intensities were obtained as a function of Vibrio parahaemolyticus concentration (FIG. 15B). The titration curve was fit to a modified Langmuir equation to obtain LOD of the test. The fit resulted in a LOD of 4.0×10⁵ CFU/mL (R²=0.997).

Performance of the Test in Hemolymph

Because operation of the test in real settings would require deployment in oysters, we ran the assay in oyster hemolymph. Hemolymph was obtained from oysters and filtered to remove any bacteria present in the real samples (FIG. 16A). Single colonies from TCBS plates were grown in SLB broth and determined the concentration of the new stocks in SLB plates. Bacteria were spiked into filtered hemolymph at concentrations ranging from 0 to 3.1×10⁷ CFU/mL to determine the test LOD in hemolymph. Accumulation of the NP-Ab conjugates at the tests area only occurred when Vibrio parahaemolyticus was present in the sample and increased the gray intensity with higher bacteria concentrations (FIG. 16B). This denoted that this complex biological fluid did not trigger non-specific binding between the nanoparticles and the immobilized antibody. The titration curve fitted into a modified Langmuir equation led to a LOD of 6.0×10⁵ CFU/mL (R²=0.996) (FIG. 16C). The pathogenic concentration of Vibrio parahaemolyticus is reported at 10⁸ cfu per serving in hemolymph for an ID=50.36 Given the fact that the volume of hemolymph in an oyster is on the order of ˜1 mL, the optimized test can identify the presence of Vibrio parahaemolyticus directly in hemolymph below this threshold. Furthermore, this challenging matrix of the oyster hemolymph did not impact the LOD of the test, leading to detection limits comparable to the standard solution used to develop it. More importantly, these results sustain that the designed test does not require any sample preparation, which eases its use in the field by non-experts.

Isolation of Negative Controls from Hemolymph

Hemolymph contains a wide variety of bacteria 37 that may result in false positives in the present test. Different bacteria species were isolated from hemolymph to explore the cross reactivity of the paper-based assay following a single colony purification method (Experimental Section). Sequencing for the conserved 16S rRNA gene was performed to identify them and concentration was determined in SLB plates prior to running the tests. Two purified samples were identified as Vibrio splendidus and Marinomonas. Vibrio splendidus is a non-pathogenic Vibrio species that is common in coastal waters but grows optimally at colder temperatures compared to Vibrio parahaemolyticus. Thus, it is critical to understand the specificity of the test against this close-related bacterium to better address its performance with environmental samples.

Study of Cross Reactivity with Negative Controls

We ran these purified negative controls from hemolymph as well as Escherichia coli. Similarly to the previous samples, each bacteria was grown in SLB broth and determined their concentration in SLB plates. The obtained pellets were spiked in hemolymph and run as previously described.

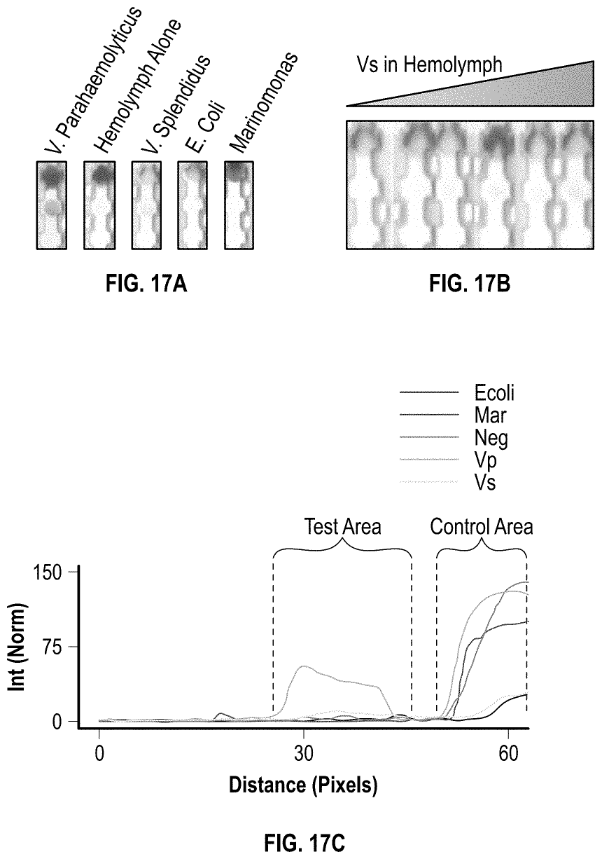

Vibrio splendidus resulted in a faint signal at the test line when run at a concentration of 6.07×107 CFU/mL (FIG. 17A), showing that the antibodies were able to pair with this other Vibrio species. Vertical line scans across the strips showed that the gray intensity in the test line for Vibrio splendidus was 6×less intense compared to Vibrio parahaemolyticus but greater than the other negative controls (FIG. 17C). This indicated that the Ab binding to this other non-pathogenic bacterium is lower compared to the reference Vibrio parahaemolyticus. To better address this binding, a titration curve was run with Vibrio splendidus spiked in filtered hemolymph (FIG. 17B). The test area intensity remained low across the Vibrio splendidus concentrations, and due to the low signal, an LOD could not be reliably quantified for these non-pathogenic bacteria (FIG. 17B). This confirmed that the antibody did not have as high an affinity for this non-pathogenic species.

We also examined the response of the assay against different negative controls spiked in hemolymph. No signal was observed at the test line while a red spot was obtained for all of them at the control line. This confirmed that the absence of signal at the test line was due to the inability of the NPs to bind to the negative controls (FIG. 17A).

Validation of the Test with Environmental Samples

We then investigated the performance of the developed test with environmental strains. Oysters were collected from coastal sites and Vibrio parahaemolyticus was isolated from hemolymph following a protocol selective for this Vibrio species, even when found at low concentrations (Experimental Section). 27 This protocol allowed us to obtain 24 environmental PCR-confirmed Vibrio parahaemolyticus isolates that were kept at −80° C. until use.

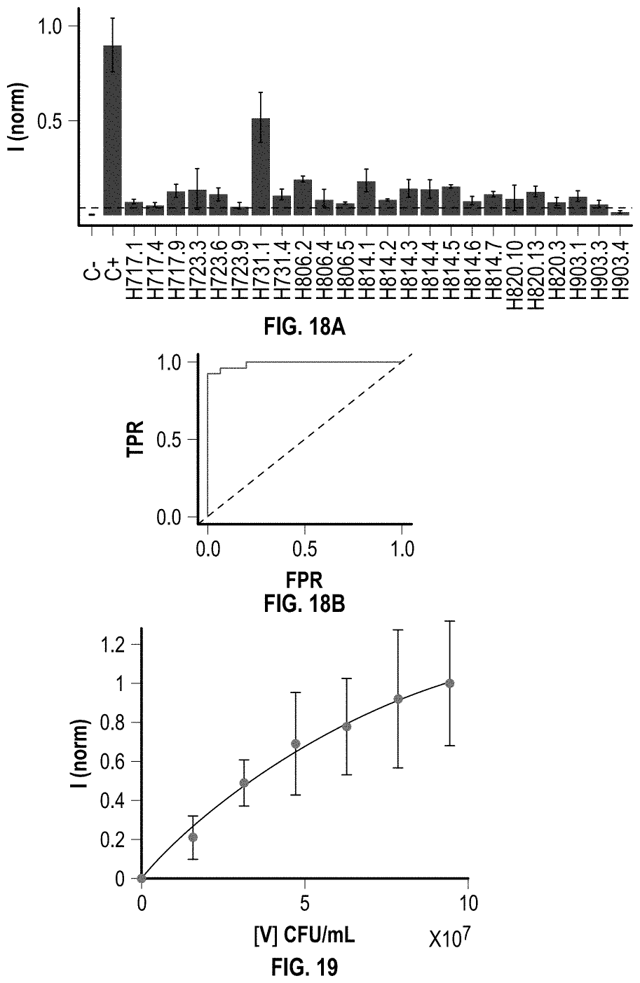

The environmental isolates and the reference Vibrio parahaemolyticus were grown for 5 h at 30° C., spiked in sterile 1×PBS and run through the tests. Six samples were chosen arbitrarily to determine the concentration range of the isolates, which was found to be between 9.4×10⁷ and 1.7×10⁸ CFU/mL. The concentration of the reference Vibrio parahaemolyticus was 1.8×10⁷ CFU/mL. Gray value intensities of the test lines were at least five and ten times greater than the standard deviation and average obtained for the negative control, respectively (FIG. 18A). Test line intensities varied among the environmental isolates probably due to the distinct binding capability of the antibodies against the isolated strains. All environmental isolates gave rise to gray intensities at least 1.7 times lower than the one obtained for the reference Vibrio parahaemolyticus from ATCC grown at the same conditions.

Image Analysis Limit of Detection

For the Limit of Detection (LOD) analysis, five independent replicates were run at different concentrations of bacteria ranging from 0 to 1.8×10⁷ CFU/mL. Gray values of the test area were obtained by subtracting the test intensity of the background and normalized with the following equation:

$${GI}_{n} = \frac{GI - {GI}_{0}}{{GI}_{max} - {GI}_{0}} ,$$

where GI is the gray intensity at a given concentration, GI0 is the gray intensity of the blank and GImax is the gray value at saturation. These values at different concentrations (GIn) were plotted and fitted in a Langmuir equation using a Matlab script (R2018a). The LOD of the test was calculated as the concentration at which the test showed a signal 3×the value of the standard deviation of the blank. The fit resulted in a LOD of 4.0×10⁵ CFU/mL (R²=0.997).

The LOD of the test in environmental samples was obtained for the isolate H731.1, which gave rise to higher intensities. The isolate was run at concentrations between 0 and 9.4×10⁷ CFU/mL and resulted in a LOD of 4.7×10⁶ CFU/mL (R²=0.993) (FIG. 19) (Tam, et al. Journal of Immunoassay and Immunochemistry 2017, 38, 355-377). This confirms that the binding of the Ab pair against environmental Vibrio parahaemolyticus is weaker than the ATCC reference, but it is still below the dose with 50% probability to cause food borne infection (ID50) reported in literature (Quantitative Risk Assessment on the Public Health Impact of Pathogenic Vibrio Parahaemolyticus in Raw Oysters; Food and Drug Administration: 2005).

Image Analysis ROC Curve

The test was validated with the PCR-identified Vibrio parahaemolyticus isolates obtained from hemolymph and run in sterile PBS in duplicates. The negative tests were also run in 1×PBS and were added to the data set used to determine the sensitivity and specificity of the paper-based assay (ranging from −0.06 to 64.9). Gray intensity values at the test line and background outside of the test area were measured using ImageJ and subtracted the background for each test. The ROC curve and the area under the curve (AUC) were obtained with the R libraries plotROC and ggplot2 (RStudio Version 1.0.153). The script varies the theoretical gray intensity threshold and classifies the tests as positive if they are found above this threshold. With the PCR-validated samples, TPR (sensitivity) and FPR (1-specificity) were calculated and plotted to obtain the ROC curve. The resulting area under the curve determines the performance of the test where 1.0 represents a perfect test and 0.5 a random indicator. The area under the curve for the tests was 0.989 (FIG. 18B). The optimal cut-off value was obtained as the one that lead to the highest value for the sum of specificity and selectivity. Thus, the optimal performance of the test was obtained with a cutoff of 2.44 leading to a sensitivity of 0.96 and specificity of 1.00. The optimal cutoff was also used to discriminate negative from positive tests from this paper-based assay. From the environmental data set only one Vibrio parahaemolyticus sample was found to be negative (H903.4), in other words, one false negative was obtained.

### Example 2: Quorum Sensing Line to Detect Activeness of Vibrio

Construction of pUC19-Sensor Plasmid

The Gibson Assembly® Cloning Kit (New England Biolabs® #E5510), was used to assemble the PUC19-Sensor Plasmid.

DNA fragments were prepared including lux box and luxR via PCR using the conditions described in Table 3, prior to undergoing purification.

TABLE 3
PCR Conditions

| | | | Number of |
|---|---|---|---|
| Step | Temperature | Duration | Cycles |
| Initial denaturation | 98° C. | 30 seconds | 1 cycle |
| Amplification | 98° C. | 10 seconds | 25-30 cycles |
| | Primer Tm (60° C. | 20 seconds | |
| | to 70° C.) | | |
| | 72° C. | 30 seconds per kb | |
| Final extension | 72° C. | 5 minutes | 1 cycle |
| Hold | 4° C. | — | 1 cycle |

The pUC19 plasmid (2686 bp) and DNA inserts for and luxR (932 bp) were incubated at 50° C. for 1 hour with the Gibson Assembly master Mix (NEB #2611) containing a 5′ exonuclease, DNA polymerase and DNA ligase. Following assembly of pUC19 and luxR, the plasmid and luxbox (50 bp) fragment were then assembled per the Gibson assembly protocol. The resulting plasmid contained luxR, luxbox and lacZ, as the pUC19 plasmid already contained lacZ. Correct insertion of the fragments was confirmed by sequencing.

Transform Vibrio parahaemolyticus Sensor Plasmid into E. coli

Following construction of the pUC19-sensor plasmid, transformation into 5-alpha Competent E. coli (NEB #C2987) occurred, followed by plating.

The assembled plasmid was added to 5-alpha competent E. coli cells and was incubated on ice for 30 minutes. The mixture was then heat shocked at 42° C. for 1 minute, before being incubated on ice for a further 2 minutes. SOC media was added and the mixture was incubated at 37° C. while shaking for 45 minutes. The bacteria mixture was spread on LB plates with ampicillin and was incubated at 37° C. overnight. The presence of ampicillin ensured that only those bacteria that successfully integrated the plasmid would grow on the LB plates.

Test for Vibrio parahaemolyticus Quorum Sensing Molecule (AHL) Detection by Cross-Streak Method

Isolated Vibrio parahaemolyticus (Vibrio parahaemolyticus) was streaked on LB plates (0.7% NaCL) and were incubated at 37° C. for 24 hours. Xgal (5-bromo-4-chloro-3-indolyl-β-D-galactopyranoside) and ampicillin were added to the plate on either side of the Vibrio parahaemolyticus growth. E. coli colonies (transformed to contain the pUC19-sensor plasmid) were streaked on either side of the Vibrio parahaemolyticus growth and the LB plates were incubated at 37° C. for 24 hours, after which time a visible blue expression was observed.

Test for Vibrio parahaemolyticus Quorum Sensing Molecule (AHL) Detection by Paper Strip

Transformed E. coli as described above was optionally mixed with a drying protectant solution, such as 1% dextrose, 0.5% peptone and 10% gelatin, as modified from Stocker et al. (Environ. Sci. Technol. 2003; 37(2):4743-4750). 4 μL of E. coli alone or with drying protectant solution was spotted four times on to nitrocellulose paper strips, as described in Example 1. Strips were dried overnight at room temperature.

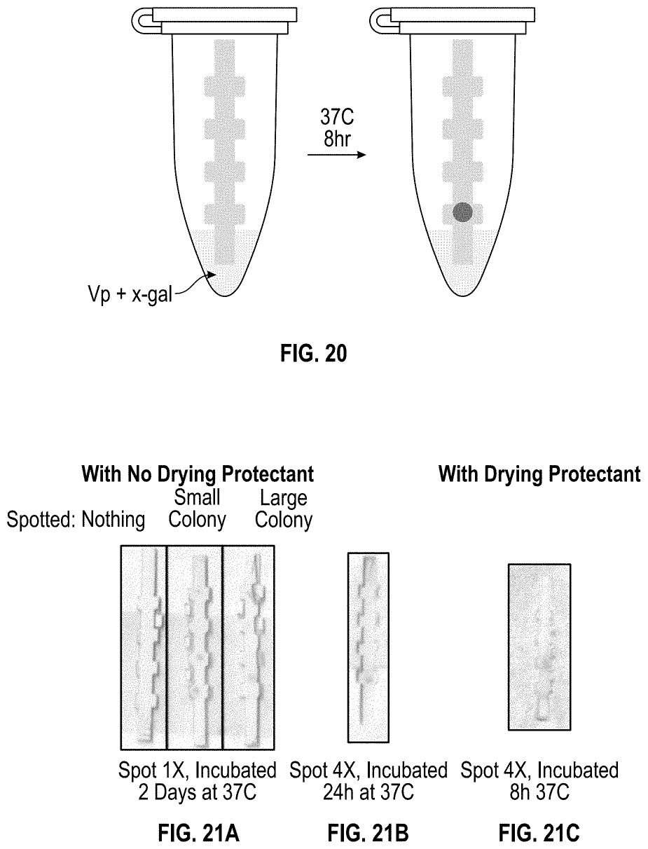

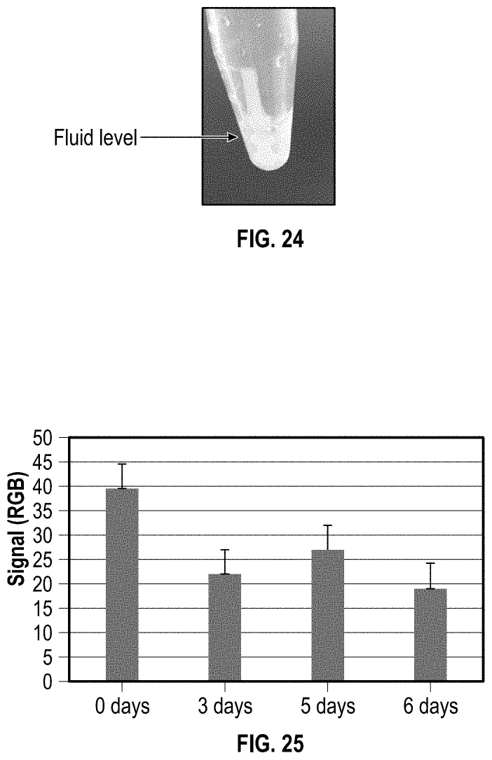

Up to 0.8 mg/mL of X-gal was add to 100 mL of solution containing Vibrio parahaemolyticus. The x-gal/Vibrio parahaemolyticus solution was placed in an Eppendorf tube, and the dried strip was added to the tube, such that the bottom of the strip was immersed in the solution as shown in FIG. 24. The tube was incubated at 37° C. for up to 8 hours as shown in FIG. 20. Strips were then removed from the tube and dried, before being imaged. Exemplary results are shown for paper strips both with and without drying protectant as shown in FIG. 21.

An additional short time course test was run. 1 μL transformed E. coli in LB media (measured OD: 1.01) was centrifuged for 90 seconds at 2000 rpm. 950 μL of supernatant was removed, and the pellet was vortexed with approximately 50-100 mL LB media to resuspend the pellet.

0.5 μL of the E. coli solution was spotted 3 times on each of three spots on a paper strip, and were allowed to dry overnight at room temperature.

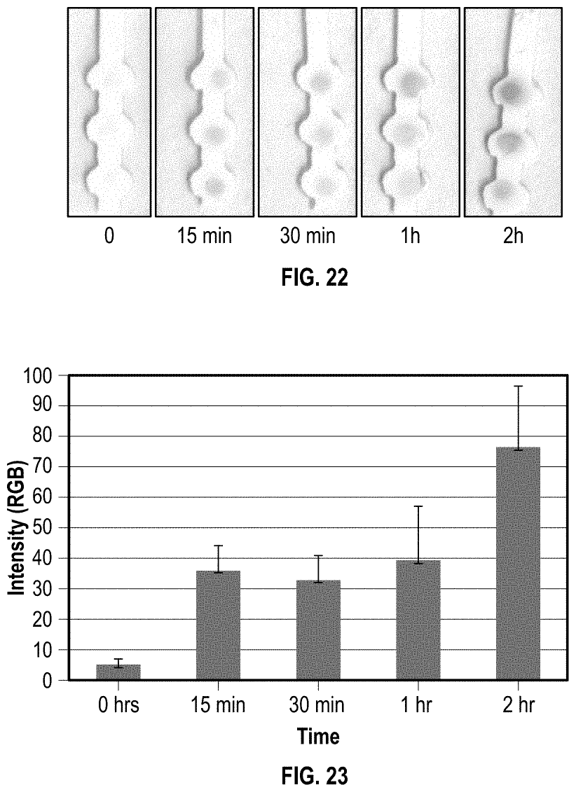

100 mL of Vibrio parahaemolyticus 17806 solution containing 0.8 mg/mL X-gal were placed in Eppendorf tubes and the paper strips were added to the tubes so that the bottom of the strip was immersed in the Vibrio parahaemolyticus solution as shown in FIG. 24. The strips were checked for the presence of a visible blue signal after 15 minutes, 30 minutes, 1 hour and 2 hours of incubation. As shown in FIG. 22, the first signal was visible after only 15 minutes incubation, and intensity of the signal increased with incubation time (FIG. 23). It was estimated that approximately 1.2×10⁷ cells were spotted onto the paper strips.

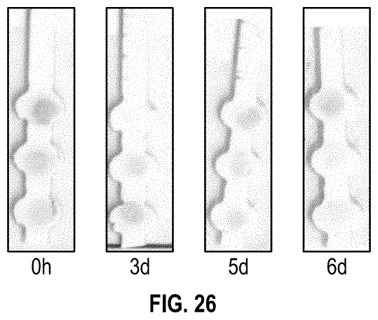

Preliminary storage results were obtained using strips prepared as above that were stored at 4° C. for up to six days. Strips were incubated with Vibrio parahaemolyticus solution as described above for 1 hour before being observed for visual signals. Results are shown in FIGS. 25 and 26.

### Example 3: Lateral Flow Immunoassay Line to Detect Pathogenicity of Vibrio (TDH/TRH)

Gold nanoparticles were prepared and characterized, Vibrio was cultured and hemolymph was extracted as described in Example 1.

Antibodies to TDH and TRH were conjugated to the nanoparticles using the same procedures as in Example 1. Paper immunoassays were prepared as described previously with TDH antibodies, TRH antibodies or both spotted onto the nitrocellulose strips. In instances where both TDH and TRH antibodies were spotted on the same strip, the antibodies were spotted in separate reaction areas of the strips. For the positive control, an anti-rabbit Fc specific antibody (add) was used in the upper spot of each strip. A blocking step was again added to prevent false positives.

Running Tests with Pure TDH/TRH

The TDH and/or TRH antigen was added to the required volume of the running medium and diluted via serial dilution. In a typical experiment 5 μL of a 1 in 100 dilution of the antigen was added to 55 μL of the running medium and homogenized. The serial dilution was conducted as 30 μL of the as made solution were placed in a tube containing 30 μL of the running medium, etc. The final 30 μL were discarded. The negative control contained 30 μL of the running medium.

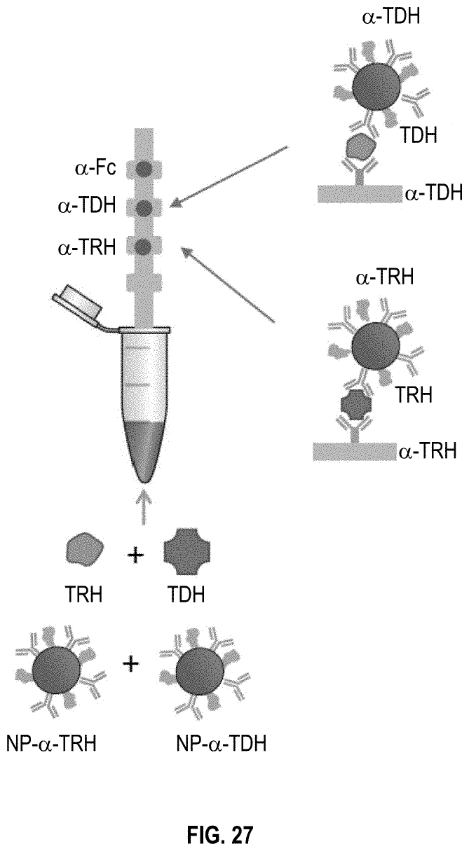

2 μL of nanoparticle-antibody conjugates were added to a solution of the antigen, homogenized and left stationary at room temperature for 20-60 minutes. After this time 15 μL of running buffer were added. The dispersion was spun in a mini centrifuge (˜1.5 krpm), vortexed and left for further 5-15 minutes. The dipsticks were placed in Eppendorfs and left to run. After the full sample volume had diffused through the paper, 1% Tween80 was ran through the dipstick to remove any non-specifically bound particles. The paper was left overnight to dry. FIG. 27 provides an exemplary diagram depicting this assay and expected results.

Running Tests with Hemolymph:

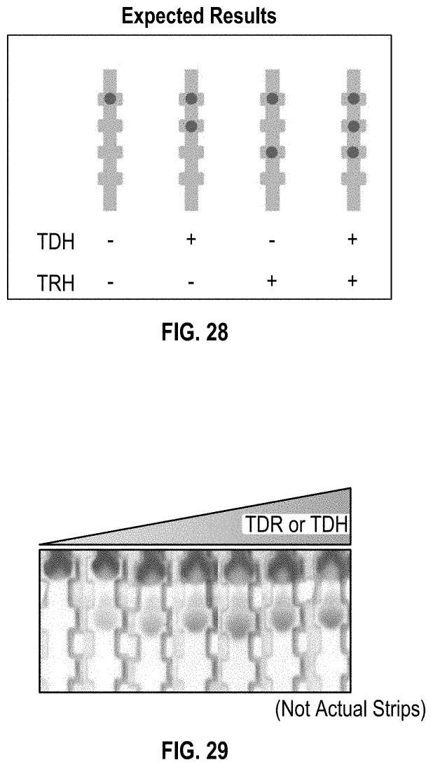

Tests were run in sequential steps to get the best results by immersing the strip in different solutions and letting the solution migrate through the strip by capillary action. First, a solution containing 12 μL of a sucrose/tween mixture (4 μL 50% w/v sucrose in water and 8 μL of 1% v/v Tween 20 in 1×PBS) and 30 μL of bacteria spiked in sterile hemolymph or PBS 1×was run. Then, the strips were washed with 50 μL PBS-Tween 20 0.1% (PBST) to avoid non-specific binding of the bacteria with the nitrocellulose. Once the strip was washed, a solution containing 12 μL of sucrose/tween, 3 μL of conjugated NPs and 30 μL of Human Serum was run to prevent false positives. A final step with 50 μL of PBST was run to wash the nitrocellulose from nonspecific binding. The strips were left to dry at room temperature and scanned for quantitative analysis. ImageJ was used to measure the gray intensity of the test area. Exemplary results are shown in FIGS. 28-29.

Image Analysis Limit of Detection

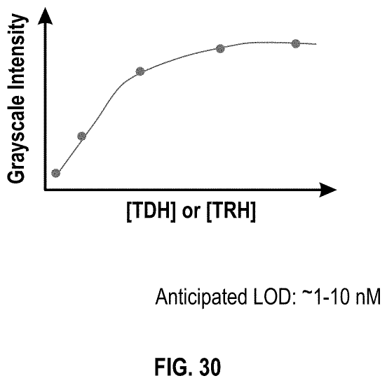

The LOD analysis will be performed as described previously in Example 1. An LOD of approximately 10 nM is anticipated, as shown in FIG. 30.

The practice of the present disclosure may employ, unless otherwise indicated, conventional techniques and descriptions of organic chemistry, molecular biology (including recombinant techniques), cell biology, biochemistry, and immunology, which are within the skill of the art.

Embodiment(s) of the present disclosure can also be realized by a computer (including cellular phone, tablet, or other device, of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like. An I/O interface can be used to provide communication interfaces to input and output devices, which may include a keyboard, a display, a mouse, a touch screen, touchless interface (e.g., a gesture recognition device) a printing device, a light pen, an optical storage device, a scanner, a microphone, a camera, a drive, communication cable and a network (either wired or wireless).

The detector interface also provides communication interfaces to input and output devices. The detector may include, for example a photomultiplier tube (PMT), a photodiode, an avalanche photodiode detector (APD), a charge-coupled device (CCD), multi-pixel photon counters (MPPC), or other. Also, the function of detector may be realized by computer executable instructions (e.g., one or more programs) recorded on a Storage/RAM.

In one embodiment, one or more of the computer system, apparatus or detector functions may be within a cellular telephone, tablet or other mobile device.

### Definitions

In referring to the description, specific details are set forth in order to provide a thorough understanding of the examples disclosed. In other instances, well-known methods, procedures, components and circuits have not been described in detail as not to unnecessarily lengthen the present disclosure.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A bacteria test device, comprising:
at least one test strip comprising at least one porous carrier and having a first end comprising a sample inlet configured to receive a liquid sample, a second end, and a longitudinal axis;
a conjugation zone disposed on the porous carrier adjacent to the first end, comprising a plurality of detection conjugates comprising nanoparticles conjugated to antibodies that specifically bind to one or more targets selected from the group consisting of: (i) bacteria of the Vibrio species; (ii) Thermostable Direct Hemolysin (TDH) and (iii) TDH-Related Hemolysin (TRH);
a first detection zone disposed on the porous carrier between the conjugation zone and the second end, configured to detect the presence of bacteria of the species Vibrio, comprising a plurality of immobilized antibodies that bind specifically to bacteria of the Vibrio species;
a second detection zone disposed on the porous carrier between the conjugation zone and the second end, configured to determine whether any bacteria present is active, comprising E. coli transformed with a quorum sensing plasmid specific to bacteria of the Vibrio species; and,
a third detection zone disposed on the porous carrier between the conjugation zone and the second end, configured to determine whether any bacteria present is virulent, comprising a plurality of immobilized antibodies that bind specifically to at least one of TDH and TRH.

2. The bacteria test device of claim 1, additionally comprising one or more control test zones disposed between the conjugation zone and the second end, wherein the control test zone comprises a plurality of antibodies that bind specifically to the Fc portion of an antibody.

3. The bacteria test device of claim 1, further comprising an absorbent sample pad disposed at and in fluid communication with the first end of the test strip.

4. The bacteria test device of claim 1, further comprising a wick disposed at the second end of the test strip.

5. The bacteria test device of claim 1, wherein the antibodies conjugated to nanoparticles are identical to the antibodies immobilized in the first and third detection zones.

6. The bacteria test device of claim 1, wherein the third detection zone comprises two sub-detection zones, the first sub-detection zone comprising immobilized antibodies to TDH, and the second sub-detection zone comprising immobilized antibodies to TRH.

7. The bacteria test device of claim 1, wherein the at least one test strip comprises a single porous carrier having a first end configured to receive a sample, a conjugation zone, and the first, second and third detection zones.

8. The bacteria test device of claim 1, wherein the at least one test strip comprises three porous carriers configured such that they are in fluid communication at each of the first ends, such that the three porous carriers jointly receive the fluid sample at the first end such that the three porous carriers are in fluidic communication with the sample inlet.

9. The bacteria test device of claim 8, wherein the three porous carriers are connected to each other at their first ends, and are separate from each other at their second ends.

10. The bacteria test device of claim 1, wherein the nanoparticles are gold nanospheres.

11. The bacteria test device of claim 1, wherein the nanoparticles are surface modified with polyethylene glycol (PEG).

12. The bacteria test device of claim 1, wherein the porous carrier is nitrocellulose.

13. The bacteria test device of claim 1, wherein the plurality of antibodies that bind specifically to bacteria of the species Vibrio are anti-V. parahaemolyticus or anti-V. vulnificus antibodies.

14. The bacteria test device of claim 1, wherein the liquid sample is oyster hemolymph.

15. The bacteria test device of claim 1, wherein the quorum sensing detection zone detects the presence of acyl-homoserine lactone (AHL) in the sample.

16. The bacteria test device of claim 1, wherein the quorum sensing plasmid comprises the genes luxbox, and luxR.

17. The bacteria test device of claim 16, wherein the transformed E. coli comprises the genes luxbox and luxR.

18. The bacteria test device of claim 17, wherein the presence of AHL acyl-homoserine lactone (AHL) in the sample induces the transformed E. coli to produce β-galactosidase.

19. The bacteria test device of claim 15, wherein:
(i) the quorum sensing detection zone additionally comprises a chromogenic or luminescent substrate that is sensitive to the presence of AHL; or,
(ii) a chromogenic or luminescent substrate that is sensitive to the presence of AHL is added to the device with the sample.

20. The bacteria test device of claim 19, wherein the chromogenic or luminescent substrate is X-gal.

21. The bacteria test device of claim 19, wherein the β-galactosidase cleaves the chromogenic or luminescent substrate to produce a visible signal.

22. The bacteria test device of claim 1, wherein the quorum sensing detection zone additionally comprises a drying protectant solution.

23. A bacteria test kit comprising:
at least two tests selected from the group consisting of:
(i) an assay to detect the presence of bacteria in a sample;
(ii) an assay to detect if the bacteria in the sample is active or not; and
(iii) an assay to detect the pathogenicity of the bacteria in the sample;
wherein, the results from the at least two tests are combined to determine whether harmful bacteria are present
wherein the bacteria are of the species Vibrio.

24. The bacteria test kit of claim 23, wherein the test kit detects the presence of harmful bacteria in a sample, wherein the sample comprises aquaculture products or water.

25. A method of detecting a pathogen in a test sample, comprising:
contacting the first end of the test device of claim 1 with a test sample under conditions allowing wicking of the sample through the first, second and third detection zones; and detecting the presence or absence of a positive test signal at each of the first, second and third detection zones;
wherein the sample has tested positive for the presence of harmful bacteria if any of the following results are obtained:
(1) all three tests are positive;
(2) tests for the presence of Vibrio and the activity of bacteria are positive;
(3) test for the virulence of bacteria is positive;
(4) tests for the activity of bacteria and virulence of bacteria are positive; or,
(5) tests for the presence of Vibrio and virulence of bacteria are positive.

26. The method of claim 25, additionally comprising:
obtaining one or more image(s) of the at least one porous carrier,
analyzing the one or more image(s) to determine whether each detection zone is positive or negative;
determining the output of the test based on the analysis of each detection zone.

27. The method of claim 25, wherein the tests located in the first, second, and third detection zones are run in parallel, in sequence, or in a combination thereof.

28. The method of claim 27, wherein the detecting of a pathogen is interrupted prior to completion and a positive result is provided upon a determination that the test for the virulence of bacteria is positive.


SEQUENCE LISTING (4 sequences)

SEQ ID NO: 1 - DNA, 17 bp, Artificial
  Primer 8F
cctacgggaggcagcag

SEQ ID NO: 2 - DNA, 17 bp, Artificial
  Primer 534R
attaccgcggctgctgg

SEQ ID NO: 3 - DNA, 41 bp, Artificial
  Primer Up1E
  misc_feature (27)..(27): n is a, c, g, or t
  misc_feature (30)..(30): n is a, c, g, or t
  misc_feature (33)..(33): n is a, c, g, or t
gaagtcatcatgaccgttctgcaygcnggnggnaarttyra

SEQ ID NO: 4 - DNA, 44 bp, Artificial
  Primer UP2AR
  misc_feature (27)..(27): n is a, c, g, or t
  misc_feature (33)..(33): n is a, c, g, or t
  misc_feature (39)..(39): n is a, c, g, or t
agcagggtacggatgtgcgagccrtcnacrtcngcrtcngycat